(19) United States
(12) Patent Application Publication
(10) Pub. No.: US 2007/0112032 A1
(43) Pub. Date: May 17, 2007

(54) Prevention and/or reduction of photoreceptor degeneration with retinoids

(76) Inventors: Scott M. Whitcup, Laguna Hills, CA (US); Roshantha A. Chandraratna, Laguna Hills, CA (US); Ronald K. Lai, Irvine, CA (US)

Correspondence Address: ALLERGAN, INC., 2525 DUPONT DRIVE, T2-7H, IRVINE, CA, 92612-1599, US

(21) Appl. No.: 10/580,879

(22) PCT Filed: Nov. 30, 2004
(86) PCT No.: PCT/US04/39987

§ 371 Date: May 25, 2006

Related U.S. Application Data
(60) Provisional application No. 60/526,505, filed on Dec. 2, 2003.

(51) Int. Cl. A61K 31/4436

(52) U.S. Cl. 514/337

(57) ABSTRACT

The present invention provides a method for reducing and/or preventing degeneration of photoreceptors in the eye of a human caused by radiation in the visible range which comprises administering to said mammal a retinoid compound having RAR_{β} and/or RAR_{δ}-selective agonist activity.

### BACKGROUND OF THE INVENTION

1. Field of the Invention

This invention relates to administering RAR_{β} and/or RAR_{δ}-selective retinoid agonists to a human to prevent and/or reduce photoreceptor damage caused by visible light, e.g. blue light.

2. Background of the Related Art

It has been observed that isotretinoin (13-cis retinoic acid or ACCUTANE®) can protect photoreceptors of rats and mice from light damage. (See Sparrow, PNAS, Apr. 15, 2003, vol. 100, no. 8, 4353-4354. See also Seiving, et al, PNAS, Feb. 13, 2001, vol. 98, no. 4, 1835-1840.) However, isotretinoin is well known to cause birth defects and is a non selective retinoid, i.e. it is not retinoid receptor subtype selective.

Tazarotene is a RAR_{β} and RAR_{δ}-selective retinoid agonist which has been used for treating psoriasis and/or acne. (See U.S. Pat. No. 5,089,509.) Tazarotene and other related retinoids are disclosed for treating various other diseases and conditions which are responsive to treatment with retinoid compounds. (See U.S. Pat. Nos. 5,750,693; 6,090,826 and 6,344,463.) Also, it has recently been disclosed that tazarotene and certain other retinoid agonists are useful in preventing the proliferation of retinal pigment epithelium following surgery or trauma or resulting from ocular diseases associated with choroidal neovascularization, such as age-related macular degeneration and histoplasmosis syndrome. (See U.S. Pat. Nos. 5,824,685; 6,075,032; 6,071,924; 6,372,753; 5,437,291 and 5,674,205.)

### BRIEF SUMMARY OF THE INVENTION

This invention provides a method for reducing and/or preventing degeneration of photoreceptors in the eye of a mammal caused by radiation in the visible range e.g. blue light, which comprises administering to said mammal a retinoid compound having RAR_{β} and/or RAR_{δ}-selective agonist activity. In particular, the invention provides a method of treating diseases and conditions resulting from or caused by exposure to visible radiation, especially radiation in the blue band of the visible spectrum, e.g. radiation of about 480 nm. Such diseases or conditions include, but are not limited to non-exudative age related macular degeneration (ARMD), exudative age related macular degeneration (ARMD), choroidal neovascularization, diabetic retinopathy, central serous chorioretinopathy, cystoid macular edema, diabetic macular edema, myopic retinal degeneration, acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (MEWDS), ocular sarcoidosis, posterior scleritis, serpiginous choroiditis, subretinal fibrosis and uveitis syndrome, Vogt-Koyanagi-Harada syndrome, punctate inner choroidopathy, acute posterior multifocal placoid pigment epitheliopathy, acute retinal pigment epitheliitis, acute macular neuroretinopathy, diabetic retinopathy, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, Eales disease, sympathetic ophthalmia, uveitic retinal disease, retinal detachment, trauma, laser, photodynamic therapy, photocoagulation, hypoperfusion during surgery, radiation retinopathy, bone marrow transplant retinopathy, proliferative vitreal retinopathy and epiretinal membranes, ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, ocular syphilis, ocular tuberculosis, diffuse unilateral subacute neuroretinitis, myiasis, retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, x-linked retinoschisis, Sorsby's fundus dystrophy, benign concentric maculopathy, Bietti's crystalline dystrophy, pseudoxanthoma elasticum, retinal detachment, macular hole, giant retinal tear, retinal disease associated with tumors, congenital hypertrophy of the retinal pigment epithelial (RPE), posterior uveal melanoma, choroidal hemangioma, choroidal osteoma, choroidal metastasis, combined hematoma of the retina and retinal pigmented epithelium, retinoblastoma, vasoproliferative tumors of the ocular fundus, retinal astrocytoma and intraocular lymphoid tumors.

Preferably, the retinoid compound is selected from the group consisting of tazarotene, i.e. ethyl-6-[2-(4,4-dimethyl-thiochroman-6-yl)ethyl]nicotinate, tazarotenic acid and other lower alkyl esters of tazarotenic acid, e.g. C₂-C₆ alkyl esters of tazarotenic acid, such as methyl 6-[2-(4,4-dimethyl-thiochroman-6-yl)ethyl]nicotinate, i-propyl 6-[2-(4,4-dimethyl-thiochroman-6-yl)ethyl]nicotinate, n-butyl 6-[2-(4,4-dimethyl-thiochroman-6-yl)ethyl]nicotinate, etc.

### BRIEF DESCRIPTION OF THE DRAWING FIGURES

FIG. 1 shows the effect of the exposure of test rats to blue light, at a wavelength of 480 nm. In particular, this Figure shows that the photoreceptor layer of the test subjects is badly damaged.

FIG. 2, in comparison to FIG. 1, shows the protective effect to the photoreceptor layer of test rats dosed with retinoids or brimonidine.

FIG. 3 shows the protective effect to the photoreceptor layer of the test rats dosed with an RAR agonist or an RXR agonist as measured by ERG.

FIG. 4 shows the relative response of the measured ERG of the photoreceptor layer of the test rats dosed with retinoids or brimonidine.

FIG. 5 shows the loss of protective effect of an RAR agonist when dosed in combination with an RAR antagonist.

### DETAILED DESCRIPTION OF THE INVENTION

Tazarotene has been used for treating acne and psoriasis and other diseases that are known to be responsive to treatment with retinoids. Also, it has recently been disclosed that tazarotene and other retinoid agonists are useful in preventing the proliferation of retinal pigment epithelium following surgery or trauma or resulting in ocular diseases associated with choroidal neovascularization, such as age-related macular degeneration and histoplasmosis syndrome.

It has now been surprisingly found that tazarotene may be used to treat diseases and/or conditions of the eye caused by exposure to visible radiation, e.g. radiation in the blue band of the spectrum. While not wishing to be bound by theory, it is postulated that tazarotene is effective as a result of its ability to act as an RAR_{β} and/or RAR_{γ}-selective retinoid agonist. (The RAR_{β} and/or RAR_{γ}-selective retinoid, utilized in the method of the present invention will preferably be incapable of agonist activity at any of the RXR receptors, and have a potency of RAR_{α}/RAR_{β} of greater than 15 and/or RAR_{α}/RAR_{γ} of greater than 30 as determined according to the cotransfection assay of Example 1 of U.S. Pat. No. 6,075,032. More preferably, the retinoid utilized in the method of the present invention will have a potency of RAR_{α}/RAR_{β} of greater than 15 and RAR_{α}/RAR_{γ} of greater than 30. See Table 1 of U.S. Pat. No. 6,075,032.)

A preferred embodiment of the present invention is the use of tazarotene for treating age-related macular degeneration, diabetic retinopathy and/or retinitis pigmentosa resulting from such radiation by contacting the eye of a person suffering from such conditions with a therapeutically effective amount of tazarotene. A therapeutically effective amount is an amount of the active agent that is effective in achieving the desired therapeutic effect. The therapeutically effective amount depends on the administration regimen, the condition of the treated individual, etc. as known per se.

To achieve a therapeutic effect of the RAR_{β} and/or RAR_{γ}-selective retinoids in the method of the present invention, the retinoid may be administered systemically, e.g. orally, or topically, e.g. by eye drop or site-selective injection into the eye, depending on the condition to be treated, the need for site-selective treatment, quantity of retinoid to be administered, and other considerations.

The invention further relates to the use of tazarotene or other RAR_{β} and/or RAR_{γ}-selective retinoids for the preparation of an ophthalmologic compositions for the treatment of ARMD, diabetic retinopathy and/or retinitis pigmentosa. That is, tazarotene is mixed with a conventional ophthalmologically compatible vehicle, for example, aqueous solutions such as physiological salines, oil solutions, or ointments. The vehicle may contain ophthalmologically compatible preservatives such as benzalkonium chloride, surfactants such as polysorbate 80, liposomes, or polymers such as methylcellulose, polyvinyl alcohol, polyvinyl pyrrolidone and hyaluronic acid, which may be used for increasing the viscosity.

As used herein, the term “a therapeutically effective amount” of tazarotene or other RAR_{β} or an RAR_{γ}-selective retinoid agonist is an amount calculated to achieve and maintain a therapeutic level in the eye, if introduced directly into the vitreous cavity or periocular space, or in the bloodstream, if administered peripherally, over the period of time desired in a human or animal such as to be effective in treating the adverse condition. The therapeutic amount will vary with the potency of each RAR_{β} and/or RAR_{δ}-selective retinoid agonist, the amount required for the desired therapeutic or other effect, the rate of elimination or breakdown of the substance by the body once it has entered the vitreous cavity or bloodstream, and the amount of the RAR agonist in the formulation. In accordance with conventional prudent formulating practices, a dosage near the lower end of the useful range of a particular agent is usually employed initially, and the dosage is increased or decreased as indicated from the observed response, as in the routine procedure of the physician.

For administration directly into the vitreous cavity of the eye, an amount in the range between about 50 and 150 μg may be administered one or more times to achieve the desired therapeutic result. Alternatively, a combination of intravitreal and subconjunctival injection of the retinoid, either simultaneously or at spaced intervals, can be used to administer the retinoid. For intravitreal injection, it is preferred that the RAR agonist be injected into the anterior vitreous cavity using topical or retrobulbar anesthesia. In an alternative embodiment, the RAR agonist is introduced intravitreally using a drug delivery vehicle. For instance, the RAR agonist can be dissolved in a biologically inert fluid that is also useful as a mechanical tamponade to help keep the retina in place, preferably an oil such as silicone oil in which the retinoid is soluble. However, for RAR agonists having partial miscibility, a liquid other than an oil can be used.

It has been discovered that the therapeutic effects of the retinoids of this invention may be delayed in onset and reversible. Therefore, it may be advantageous to administer the retinoids utilizing a method of a slow release, for instance by intravitreal injection of the dose of retinoid encapsulated in a microvesicle, such as a liposome, from which the dose is released over the course of several days, preferably between about 3 to 20 days. Alternatively, the drug can be formulated for slow release, such as incorporation into a slow release polymer from which the dosage of drug is slowly released over the course of several days, for example from 2 to 30 days. The slow release formulation may be placed in the eye by intravitreal, subconjunctival, periocular, intrascleral or subretinal injection. The retinoid may be incorporated into a bioerodible polymer such as a polylactic acid-glycolic acid copolymer, e.g. Oculex®.

The ophthalmologic compositions of this invention may be administered in a number of ways. By one mode of administration, said ophthalmologic composition is applied topically onto the eye. For topical application, said ophthalmologic composition may be formulated with a vehicle that is compatible with the eye and preferably such that facilitates penetration of tazarotene into the eye. For such mode of application, said active agent may be formulated in the form of eyedrops (in which the tazarotene or other RAR_{β} and/or RAR_{γ}-selective retinoid agonist is dissolved in a physiological solution), in the form of ointments, in the form of a liposome solution, etc.

It is contemplated that the dosing levels of tazarotene as used in the eye drops of the present invention would be adjusted as necessitated by lack of response, speed of response needed, strength of tazarotene solution, etc.

The method of the present invention may be practiced alone or in conjunction with other therapy.

The invention is further illustrated by the following examples which are illustrative of specific modes of practicing the invention and are not intended as limiting the scope of the appended claims.

Adult male abino Sprague-Dawley rats (weight 400±30 g) were used for the following examples. After 18 hours of dark adaptation, animals were housed in specially designed acrylic cages and exposed to high intensity (12000 LUX) of blue fluorescent light (480 nm) for 8 hours. The light intensity was measured with a digital light meter. Each animal was housed separately. The room was maintained at 73° F. throughout the experiment. Animals were orally dosed with the appropriate retinoid or positive control, i.e. brimonidine, for 5 days with the last dose administered 2 hours before blue-light exposure. After the light exposure, the animals were kept in the dark room and recovered for an additional 5 days. Retinal function was evaluated with flash ERG analysis. Retinal structure was assessed by histology.

### EXAMPLE 1

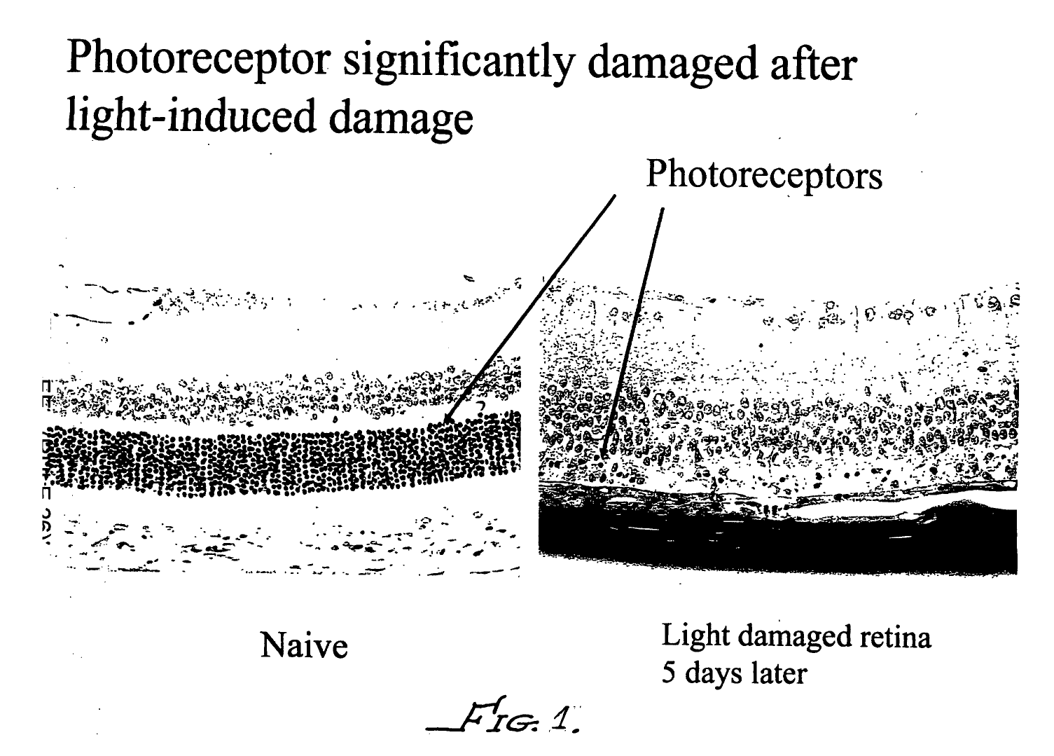

As shown in FIG. 1, the photoreceptor layer is badly damaged by exposure to blue light in this experiment where the animals are not dosed with a retinoid or other neuroprotective agent.

### EXAMPLE 2

The following retinoids were evaluated for preventing damage to the photoreceptor layer of rats subjected to exposure to blue light.

Retinoid compound tested/receptor selectivity/dose.

Tazarotene/(RAR agonist)/3 mg/kg/day

Compound A/(RXR agonist)/10 mg/kg/day

Compound B/(RXR antagonist)/50 mg/kg/day

Compound C/(RAR antagonist)/3 mg/kg/day

Compound A

3,7-Dimethyl-6(S),7(S)-methano-7-[1,1,4,4-tetramethyl-1,2,3,4-tetrahydronaphth-7-yl]-2E, 4E-heptadienoic acid
   Compound B
(2E,4E,6E)-7-(3,5-Diisopropyl-2-propoxy-phenyl)-6-fluoro-3-methyl-nona-2,4,6-trienoic acid
   Compound C
4-2(6-(2,2-Dimethyl-(1H)-4-(4-ethylphenyl)-1-benzothiopyran))ethynyl]benzoic acid

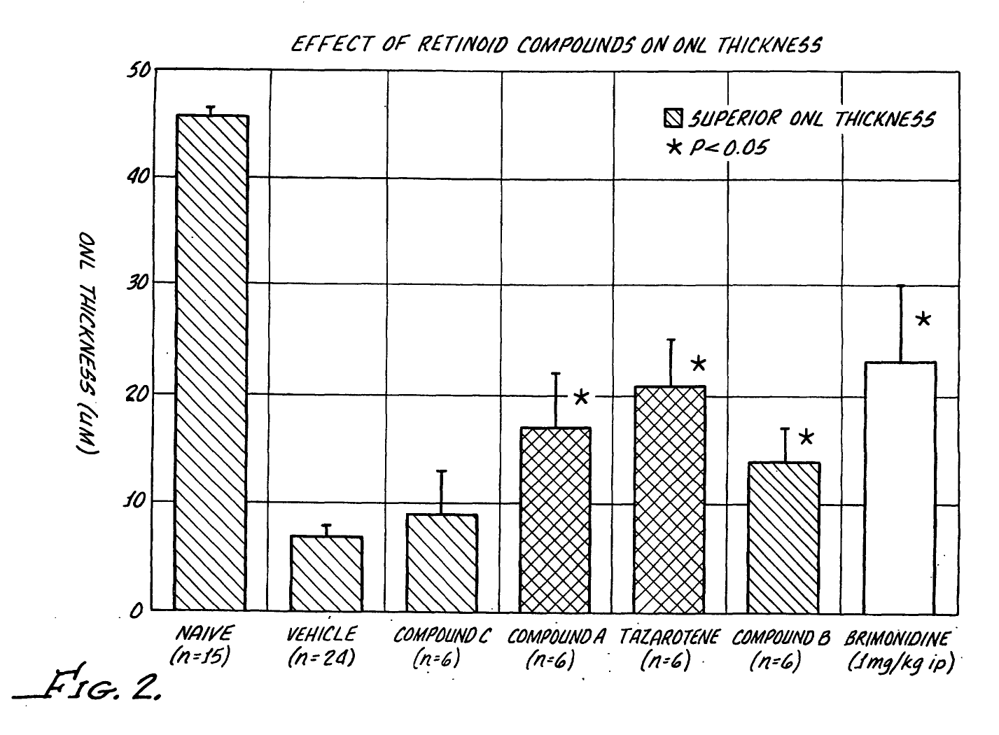

As shown in FIG. 2, the thickness of the photoreceptor layer for the animals dosed with brimonidine, a well known neuroprotective agent is much greater than the thickness of the photoreceptor layers of the animals dosed with the vehicle alone, or the RAR antagonist or the RXR antagonist. The thickness or the photoreceptor layer for the RXR agonist is greater than the photoreceptor layers of the animals dosed with the RAR or RXR antagonists but, the photoreceptor layer of the animal dosed with the RAR agonist is the best of the retinoids tested and almost equivalent in effect to brimonidine. (The RAR agonist, tazarotene, is an RAR_{β} and RAR_{δ}-selective retinioid. The RXR agonist also has some RAR agonist activity.)

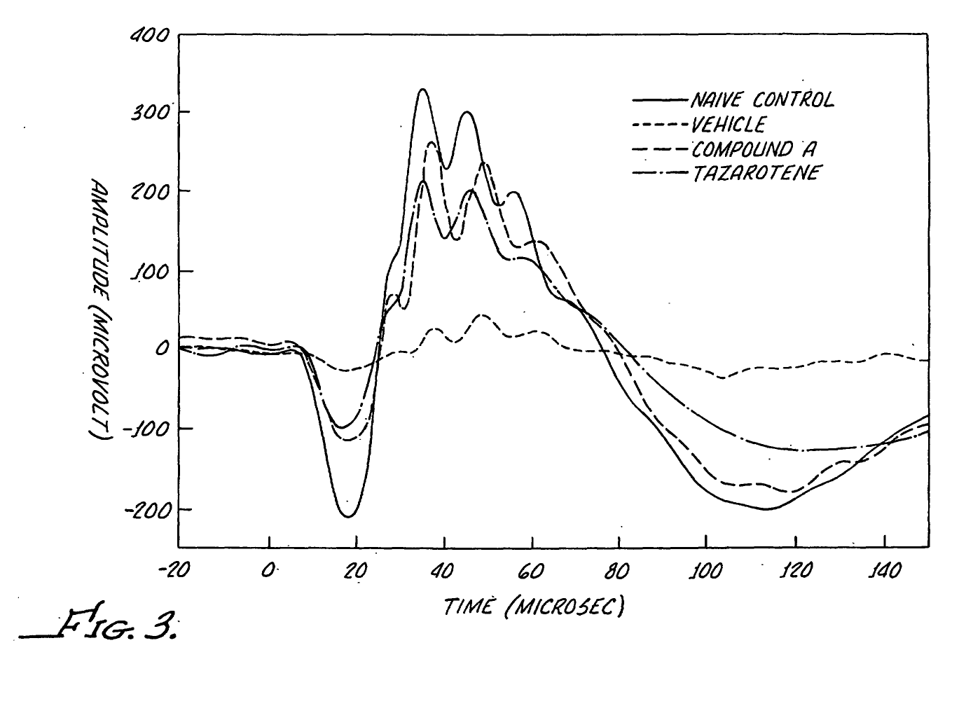

Also, as shown in FIG. 3, which is a plot of the ERG wave verses time, the RAR agonist and the RXR agonist show a protective effect to the photoreceptor layer when measured by ERG.

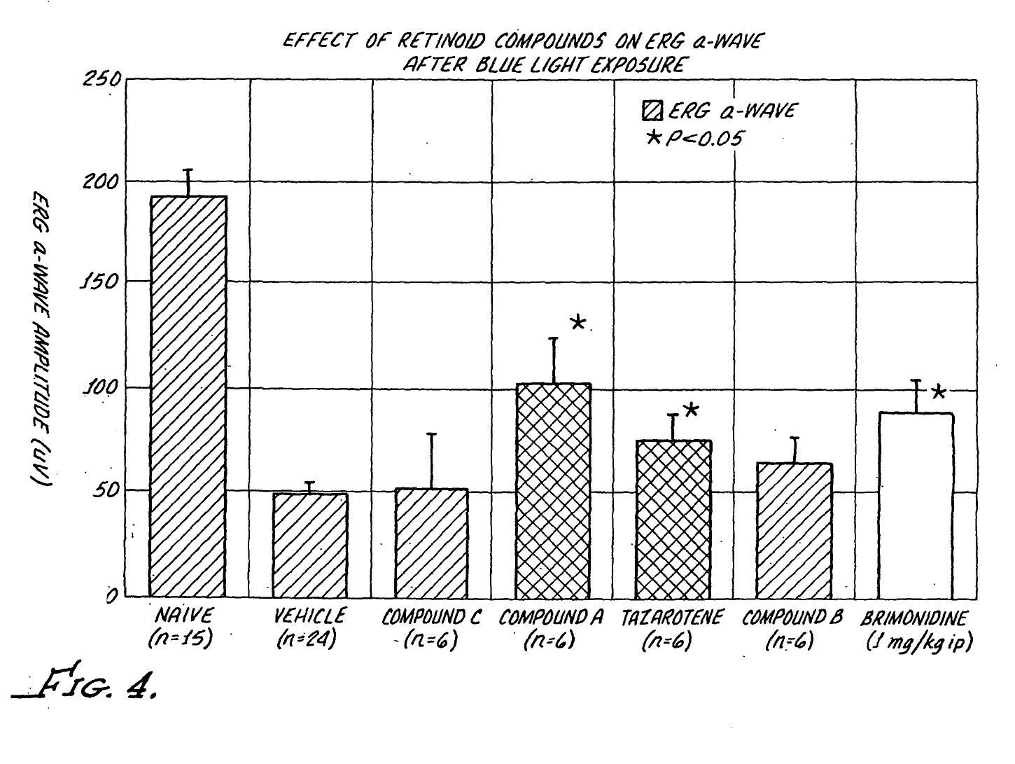

FIG. 4, shows in a bar chart the relative response of the ERG wave for the above animals after exposure to blue light.

### EXAMPLE 3

In this Example, the above experiment is repeated with the RAR antagonist, which has antagonist activity at the α, β and δ retinoid receptor subtype dosed in combination with the RAR agonist and the RXR agonist.

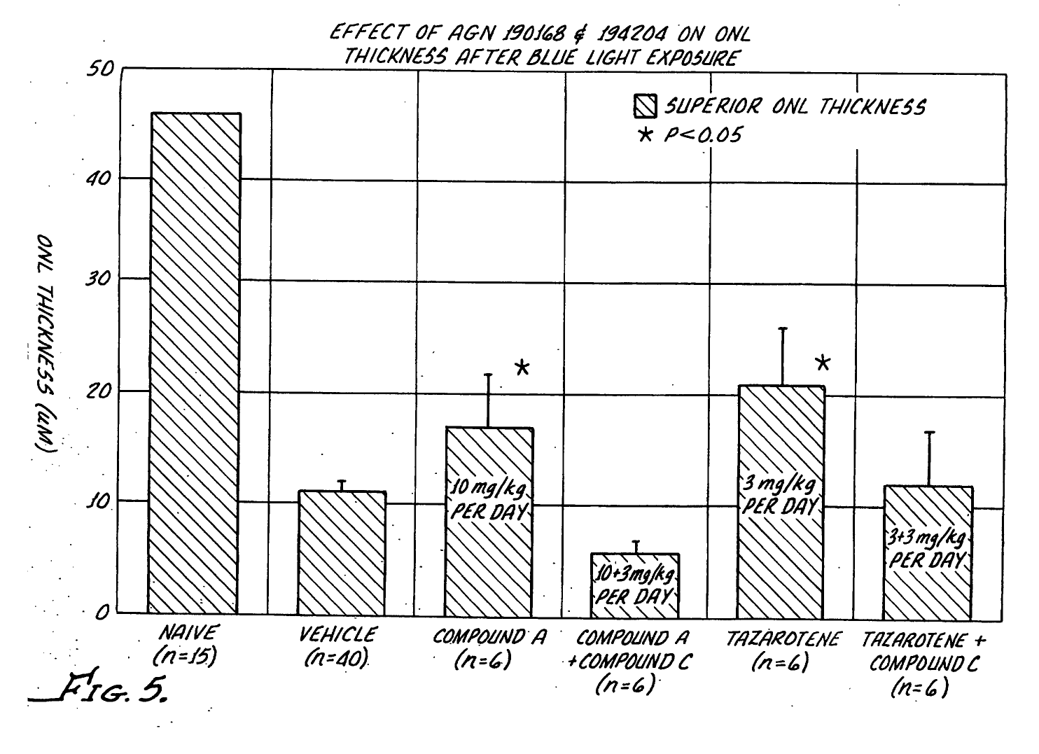

FIG. 5 shows that the RAR antagonist severely diminishes the effectiveness of both the RAR agonist and the RXR agonists, therefore demonstrating that the effectiveness of the RXR antagonist is a result of its RAR agonist activity and not its RXR antagonist activity.

### EXAMPLE 4

The following retinoid compounds were tested in the above method with the results reported as shown.

| | Protective |
|---|---|
| Retinoid Compound/Receptor Selectivity | Activity |
| Tazarotene/(RAR_{β, γ} agonist) | *** |
| Compound A/(RXR_{α, β, γ} agonist with RAR activity) | ** |
| Compound C/(RAR_{α, β, γ} antagonist) | X |
| Compound B/(RXR_{α}, β, γ antagonist) | X |
| Compound D/(RAR_{α} antagonist) | X |
| Compound E/(RAR_{α} agonist) | ** |
| Compound F/(RXR_{α, β, γ} agonist) | X |

* means a protective effect of the photoreceptor layer to damage from blue light radiation. Greater effectiveness is shown by increasing number of *'s.
X means no such protective effect.
Compound D
2-Fluoro-4-[6′-(2″,2″-dimethyl-4″-tolyl chromanyl)-8'-bromo]carbamoyl benzoic acid
Compound E
4-[(4-Chloro-3-hydroxy-5,5,8,8-tetramethyl-5,6,7,8-tetrahydro-naphthalene-2-carbonyl)-amino]-2,6-difluoro-benzoic acid
Compound F
3-Methyl-7-propyl-6(S),7(S)-methano-7[1,1,4,4-tetrametyl-1,2,3,4-tetrahydro-7-yl]-2(E),4(E)-heptadienoic acid

### EXAMPLE 5

In an example of treatment according to the preferred embodiment described above, a male patient aged 64 with blue eyes is diagnosed with age-related macular degeneration of about ten years' duration. Numerous druscen was documented in both eyes. Photographs of the fundus are obtained. Treatment with tazarotene according to the preferred method of use described herein is initiated in the left eye.

After two years of treatment, the treated eye shows no changes in visual acuity from that measured at the start of treatment. There are also no changes in the fundus, such as increases in the number or extent of the druscen, compared with the photographs obtained before the start of treatment. Thus, treatment with tazarotene according to the methods of the present invention prevents any additional effects from macular degeneration from occurring in the treated eye. This is significant because, as described above, the normal course of macular degeneration leads to a continuous, on-going loss of vision over time.

The above disclosure sets forth an embodiment of the present invention. Other arrangements or embodiments, not precisely set forth, could be practiced under the teachings of the present invention.

While the present invention has been described in the context of treating age-related macular degeneration, tazarotene may also be used for treating retininitis pigmentosa, diabetic retinopathy, ischemic retinopathy damage caused by surgery, e.g. laser or mechanical, and photodynamic therapy and any of the other diseases and/or conditions disclosed above.

Moreover, while the present invention is described for treating retinitis pigmentosa with tazarotene, the corresponding acid, i.e. tazarotenic acid may also be used, as well as other C₁ to C₆ lower alkyl esters of tazarotenic acid, e.g. the methyl and isopropyl esters of tazarotenic acid.

The above disclosure sets forth certain preferred embodiments of the present invention. Other arrangements or embodiments, not precisely set forth, could be practiced under the teachings of the present invention.

We claim:

1. A method for reducing and/or preventing degeneration of photoreceptors in the eye of a human caused by radiation in the visible range which comprises administering to said mammal a retinoid compound having RAR_{β} and/or RAR_{δ}-selective agonist activity.

2. The method of claim 1 wherein said radiation is blue light radiation.

3. The method of claim 1 wherein said retinoid compound is tazarotenic acid or a lower alkyl ester or salt thereof.

4. The method of claim 3 wherein said compound is tazarotenic acid or tazarotene.

5. The method of claim 4 wherein said compound is tazarotene.

6. A method of treating diseases or conditions in a mammal resulting from or caused by exposure to visible radiation which comprises administering to said mammal a retinoid compound having RAR_{β} and/or RAR_{δ}-selective agonist activity.

7. The method of claim 6 wherein said radiation is blue light radiation.

8. The method of claim 6 wherein said retinoid compound is tazarotenic acid or a lower alkyl ester or salt thereof.

9. The method of claim 6 wherein said compound is tazarotenic acid or tazarotene.

10. The method of claim 6 wherein said compound is tazarotene.

11. The method of claim 1 wherein said mammal has a condition selected from the group consisting of non-exudative age related macular degeneration (ARMD), exudative age related macular degeneration (ARMD), choroidal neovascularization, diabetic retinopathy, central serous chorioretinopathy, cystoid macular edema, diabetic macular edema, myopic retinal degeneration, acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (MEWDS), ocular sarcoidosis, posterior scleritis, serpiginous choroiditis, subretinal fibrosis and uveitis syndrome, Vogt-Koyanagi-Harada syndrome, punctate inner choroidopathy, acute posterior multifocal placoid pigment epitheliopathy, acute retinal pigment epitheliitis, acute macular neuroretinopathy, diabetic retinopathy, retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, sympathetic ophthalmia, uveitic retinal disease, retinal detachment, trauma, laser, photodynamic therapy, photocoagulation, hypoperfusion during surgery, radiation retinopathy, bone marrow transplant retinopathy, proliferative vitreal retinopathy and epiretinal membranes, ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, ocular syphilis, ocular tuberculosis, diffuse unilateral subacute neuroretinitis, myiasis, retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, x-linked retinoschisis, Sorsby's fundus dystrophy, benign concentric maculopathy, Bietti's crystalline dystrophy, pseudoxanthoma elasticum, retinal detachment, macular hole, giant retinal tear, retinal disease associated with tumors, congenital hypertrophy of the retinal pigment epithelial (RPE), posterior uveal melanoma, choroidal hemangioma, choroidal osteoma, choroidal metastasis, combined hematoma of the retina and retinal pigmented epithelium, retinoblastoma, vasoproliferative tumors of the ocular fundus, retinal astrocytoma and intraocular lymphoid tumors.

12. The method of claim 11 wherein said condition is age related macular degeneration.

13. The method of claim 11 wherein said condition is retinitis pigmentosa.

14. The method of claim 11 wherein said condition is diabetic retinopathy.

15. The method of claim 11 wherein said condition is surgical trauma.

16. The method of claim 11 wherein said condition is laser induced damage.
